(19) United States
(12) Patent Application Publication
(10) Pub. No.: US 2026/0174974 A1
(43) Pub. Date: Jun. 25, 2026

(54) A DISPOSABLE CASSETTE, A SUBASSEMBLY OF A MEDICAMENT DELIVERY DEVICE AND A MEDICAMENT DELIVERY DEVICE

(71) Applicant: SHL Medical AG, Zug (CH)

(72) Inventors: Daniel Säll, Nacka Strand (SE); Rasmus Renstad, Nacka Strand (SE)

(21) Appl. No.: 19/125,878

(22) PCT Filed: Oct. 20, 2023
(86) PCT No.: PCT/EP2023/079216

§ 371 Date: Apr. 30, 2025

(30) Foreign Application Priority Data
Nov. 4, 2022 (EP) 22205520.4

(51) Int. Cl. A61M 5/32; A61M 5/145; A61M 5/20; A61M 5/315

(52) U.S. Cl. CPC A61M 5/3202; A61M 5/1456; A61M 5/2033; A61M 5/31501; A61M 5/31511; A61M 5/31553; A61M 5/3204; A61M 5/326; A61M 5/3287; A61M 2005/208

(57) ABSTRACT

The present disclosure generally relates to medicament delivery devices such as autoinjectors, and particularly concerns a disposable cassette configured to accommodate a medicament container, and to a subassembly for a medicament delivery device.

### TECHNICAL FIELD

The present disclosure generally relates to medicament delivery devices such as autoinjectors or infusers, and particularly concerns a disposable cassette configured to accommodate a medicament container, and to a subassembly for a medicament delivery device.

### BACKGROUND

A number of medical conditions require injections. These days, a number of different injection devices exist, including various types of pen injectors, autoinjectors and infusion devices for large volume injections. Although many of these devices have enabled major improvements in the management of a number of medical conditions, various limitations do still exist in the current technology. Not least amongst these are the difficulties faced by patients that require frequent injections and by patients that need to inject particularly viscous drugs. Further, to increase patient convenience it is often desirable to reduce the injection frequency. In considering these problems, the applicant has appreciated that various developments could be made to help improve the medicament delivery devices on the market today, for example concerning more effective means to administer larger injection volumes, which are set out in more detail below.

### SUMMARY

An object of the present disclosure is to provide a cassette and a subassembly for a medicament delivery device which solves, or at least mitigates problems of the prior art.

According to a first aspect of the present disclosure, there is provided a disposable cassette configured to accommodate a medicament container, the cassette is attachable to a medicament delivery device for delivery of the medicament to a medicament delivery site via an infusion set attachable to the cassette, the cassette comprising: A tubular main body adapted to receive the medicament container radially inside the tubular main body, the tubular main body having a through hole at a proximal end through which an adapter of the infusion set is attachable to the medicament container.

The tubular main body comprises a radially extending protrusion on a radially outwards facing surface, the radially extending protrusion includes a proximally facing surface.

The disposable cassette is attachable to a proximal end of the medicament delivery device by insertion of the disposable cassette in a proximal receiving interface of the medicament delivery device, and a subsequent axial rotation of the disposable cassette, whereby the radially extending protrusion is configured to slide on a curved distally facing surface of the receiving interface of the medicament delivery device until the proximally facing surface engages with a distally facing surface of the receiving interface to longitudinally lock the disposable cassette in an attached position in the receiving interface.

Embodiments of the present disclosure advantageously provides for a disposable cassette that accommodates a medicament container. The disposable cassette is attachable to a medicament delivery device by insertion in the receiving interface to lock the disposable cassette in an attached position in the receiving interface. By means of the separate disposable cassette, variable medicament volumes are possible using the same medicament delivery device. Thus, the volume of the medicament container can range from smaller volumes of about 2 ml to larger volumes of about 10 ml or more depending on the size of the disposable cassette. Regardless of medicament volume, the engagement with the medicament delivery device is the same as provided by the interface between the receiving interface of the medicament delivery device and the disposable cassette.

Embodiments of the present disclosure further provides the advantage that the injection frequency can be reduced since larger medicament volumes can be administered. This avoids issues related to medicament injection of high viscosity medicament solutions if the same amount of medicament is provided in smaller volumes to in that way reduce the number of required injections.

Still further, using a disposable cassette provides for an environmentally friendly and cost-effective solution for proving several medicaments with a single device.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the components thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, “circumferentially” refer to a circumference or a circumferential direction 301 relative to an axis 102, typically a central axis extending in the direction of the longest extension of the device and/or component. Similarly, “radial” or “radially” refer to a direction 302 extending radially relative to the axis, and “rotation”, “rotational” and “rotationally” refer to rotation relative to the axis.

According to one embodiment, the disposable cassette may comprise an electronic tag attached to a distal end of the tubular main body, the electronic tag having stored data that is related to the medicament contained within the tubular main body, wherein the data is readable by an electronic reader of the medicament delivery device when the disposable cassette is in the attached position. The electronic tag advantageously enables for providing the data to the medicament delivery device to make corresponding settings.

According to one embodiment, the disposable cassette may comprise at least one distally extending protrusion that extend distally away from a distal end of the tubular main body, wherein the at least one distally extending protrusion comprise a distally facing surface that is configured to abut against and push an activation interface of the medicament delivery device in a distal direction to cause actuation of a first switch that activates the electronic reader. Thus, when the disposable cassette is fully inserted in the receiving interface, or at least sufficiently to reach the activation interface, the activation interface is advantageous pushed without any additional required action from the user, to activate the electronic reader.

According to one embodiment, the distally extending protrusion comprises a distally facing inclined surface inclined with respect to a longitudinal axis of the tubular main body, wherein the distally facing inclined surface is arranged to interact with a surface of the activation interface when the disposable cassette is rotated further. The interaction distally facing inclined surface facilitates for an axial motion of the activation interface caused by the interaction.

Thus, in one embodiment, the interaction between the distally facing inclined surface and the surface of the activation interface cause the activation interface to move distally.

Further, in one embodiment, the activation interface activates a second switch when it is moved distally by the interaction. Thus, only by rotating the disposable cassette, the activation interface activates the second switch.

Advantageously, the activation of the second switch actuates a plunger rod of the medicament delivery device for initiating a priming action of the medicament container. Priming is the action of expelling air bubbles from the syringe.

According to one embodiment, the disposable cassette may comprise a second set of radially extending protrusions on the radially outwards facing surface that are configured to engage in corresponding radially inner facing slots of the receiving interface to rotationally lock the disposable cassette in the receiving interface. The rotational locking gives an intermediate position of the disposable cassette where the electronic reader is activated but the plunger rod does not yet axially move.

According to one embodiment, the second set of radially extending protrusions may be longitudinal ribs.

According to one embodiment, the disposable cassette may comprise an activation ring removably attached radially inside the tubular body, the activation ring comprises the at least one distally extending protrusion. The electronic tag may be attached to the activation ring.

According to one embodiment, the activation ring may comprise two distally extending protrusion symmetrically arranged with respect to a longitudinal axis of the tubular main body. The symmetric arrangement provides for move evenly distributed force on the activation surface when the disposable cassette is inserted in the receiving interface of the medicament delivery device.

According to one embodiment, the disposable cassette may advantageously comprise a removable tamper seal that seals the through hole at the proximal end of the tubular main body. The tamper seal is removed by a user prior to use of the disposable cassette.

Further, the disposable cassette may advantageously comprise a plunger rod adapter fitted in the tubular main body to be pushed proximally by a plunger rod of the medicament delivery device during a medicament delivery event.

According to one embodiment, the disposable cassette may comprise an annular cavity between the tubular main body and the medicament container at the proximal end of the tubular main body, wherein the annular cavity is configured to receive a flange of the adapter of the infusion set. Hereby, in the annular cavity, improved locking possibilities for locking the infusion set to the disposable cassette are provided.

According to one embodiment, the tubular main body may comprise longitudinal ribs facing radially inwards in the cavity, the longitudinal ribs being adapted to rotationally lock the adapter. Longitudinal ribs provide a reliable way of providing a rotational lock, when engaged in a fitting groove or trench.

According to one embodiment, the disposable cassette may comprise a further protrusion on the radially outwards facing surface of the tubular main body, the further protrusion is located proximally with respect to the radially extending protrusion, the further protrusion being configured to abut against the receiving interface to limit the insertion length of the disposable cassette in the receiving interface. In this way, the disposable cassette is not inserted too far in the receiving interface.

According to one embodiment, the attachment between the disposable cassette and the receiving interface may be a bayonet coupling.

There is further provided an infusion device comprising a medicament delivery device and a disposable cassette.

According to a second aspect of the present disclosure, there is provided subassembly for a medicament delivery device comprising: a proximal receiving interface adapted to receive a cassette accommodating a medicament container, the proximal receiving interface comprising a curved distally facing surface adapted to receive and slidably guide a radially extending protrusion of the cassette in a distal direction when the cassette axially rotates relative the receiving interface until a proximally facing surface of the cassette engages with a distally facing surface of the receiving interface to longitudinally lock the disposable cassette in an attached position in the receiving interface, an activation interface comprising a proximally facing surface adapted to receive a distally extending protrusion of the cassette, wherein when the activation interface is pushed distally by the cassette it causes activation of a first switch of the subassembly to activate an electronic reader to read an electronic tag of the cassette.

According to one embodiment, the activation interface may comprise a first and second distally extending arms extending towards a respective first and second switch, where the first arm reaches and causes the activation of the first switch when the activation interface is moved distally.

According to one embodiment, the activation interface may comprise an inclined surface adapted to receive the distally extending protrusion of the cassette when the cassette is further rotated in the receiving interface, whereby an interaction between the distally facing surface of the cassette and the inclined surface of the activation interface cause the activation interface to move distally.

According to one embodiment, the distal motion of the annular activation interface may cause the second arm to reach to and active the second switch.

According to one embodiment, the distal ends of the first arm and the second arm may be asymmetric.

According to one embodiment, the subassembly may advantageously comprise a spring arranged to spring load the activation interface away from the first switch and the second switch.

There is further provided a medicament delivery device comprising the subassembly of any of the herein disclosed embodiments.

Generally, all terms used in the claims are to be interpreted according to their ordinary meaning in the technical field, unless explicitly defined otherwise herein. All references to “a/an/the member, apparatus, component, means, etc.” are to be interpreted openly as referring to at least one instance of the member, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 is a perspective view of a medicament delivery device according to embodiments of the present disclosure;

FIG. 2A is a perspective view of a disposable cassette according to embodiments of the present disclosure;

FIG. 2B is a perspective view of a proximal end of the tubular main body and an adapter of an infusion set according to embodiments of the present disclosure;

FIG. 3 is a perspective view of a receiving interface according to embodiments of the present disclosure;

FIG. 4 is a perspective view of a receiving interface according to embodiments of the present disclosure;

FIG. 5 is a perspective view of a receiving interface according to embodiments of the present disclosure;

FIG. 6 is a perspective view of an activation interface according to embodiments of the present disclosure;

FIG. 7 is a perspective view of an activation interface according to embodiments of the present disclosure;

FIG. 8 is a perspective view of the medicament delivery device with housing removed according to embodiments of the present disclosure;

FIG. 9 is a close-up view of a distal end of the disposable cassette and the receiving interface according to embodiments of the present disclosure;

FIG. 10 is a close-up view of a distal end of the disposable cassette and the activation interface according to embodiments of the present disclosure;

FIG. 11 is a close-up view of a distal end of the disposable cassette and the activation interface according to embodiments of the present disclosure;

FIG. 12 is a close-up view of a distal end of the disposable cassette and the activation interface according to embodiments of the present disclosure;

FIG. 13 is a close-up view of a distal end of the disposable cassette and the activation interface with the first switch activated according to embodiments of the present disclosure;

FIG. 14 is a close-up view of a distal end of the disposable cassette and the activation interface with the first switch activated according to embodiments of the present disclosure; and

FIG. 15. is a close-up view of a distal end of the disposable cassette and the activation interface with the second switch activated according to embodiments of the present disclosure.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art.

Like numbers refer to like members throughout the description.

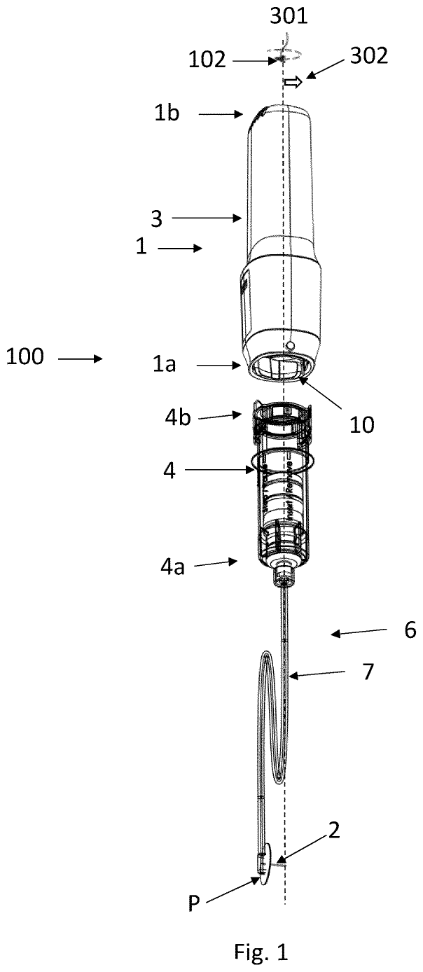

FIG. 1 shows an example of a medicament delivery device 1 such as an autoinjector of an injection device, or an infusion device 100 according to embodiments of the present disclosure. The medicament delivery device 1 is configured to expel medicament from a medicament container via a medicament delivery member such as a needle 2, to a user at a dose delivery site. The medicament delivery device 1 extends from a proximal end 1a to a distal end 1b relative to the axis 102.

The medicament delivery device 1 comprises a housing 3 and is connectable to a disposable cassette 4 having a proximal end 4a and a distal end 4b.

The disposable cassette 4 connected to an infusion set 6 comprising a flexible tube 7 connected to a through hole of the disposable cassette 4 and the proximal end 4a. At the other end of the tube 7 is the needle 2 attached via a pad P.

The disposable cassette 4 is adapted to accommodate a medicament container which can be of different volumes, for example ranging from about 1 ml to about 10 ml.

The medicament delivery device 1 comprises a receiving interface 10 configured to received and lock the disposable cassette to the proximal end 1a of the medicament delivery device 1.

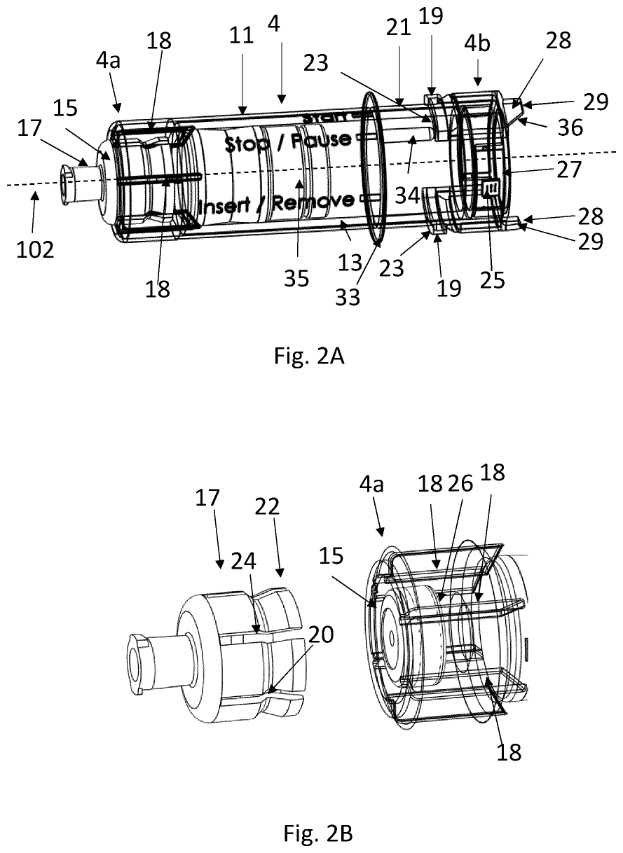

FIG. 2A is a perspective view of a disposable cassette according to embodiments of the present disclosure. The disposable cassette 4 comprises a tubular main body 11 adapted to receive the medicament container 13 radially inside the tubular main body 11. The tubular main body 11 has a through hole 15 at the proximal end 4a through which an adapter 17 of the infusion set 6 is attachable to the medicament container 13. Preferably, a removable tamper seal that seals the through hole 15 is removed prior to mounting the adapter 17 in the through hole 15.

Turning to FIG. 2B, in some embodiments, the tubular main body 11 comprises longitudinal ribs 18 facing radially inwards in a cavity between the medicament container 13 and the tubular main body at the proximal end 4a of the tubular main body 4. The annular cavity is configured to receive a flange 22 of the adapter 17 of the infusion set. The longitudinal ribs 18 are adapted to rotationally lock the adapter by engaging in longitudinal slots 24 of the flange 22. In addition, a radially inwards extending protrusion 20 of the flange 22 engage with or grips a neck 26, or crimped part, of the medicament container 13. The tubular main body 4 is adapted to allow the adapter 17 to expand when the adapter is mounted over the neck 26 of the medicament container.

Turning again to FIG. 2A, the tubular main body 11 comprises a radially extending protrusion 19 on a radially outwards facing surface 21. The radially extending protrusion 19 includes a proximally facing surface 23.

The radially extending protrusion 19, or as here exemplified as being two separate protrusions 19, extend along a circumference of the outer surface 21 so that the protrusions 19 are curved along the circumference. However, the protrusions 19 only extend along part of the circumference of the tubular body 11.

The disposable cassette 4 comprises an electronic tag 25 here attached at the distal end 4b of the tubular main body 11. The electronic tag 25 may be attached to the tubular main body 11 or to an activation ring 27 removably attached radially inside the tubular body 11. The electronic tag 25 is configured to store data that is related to the medicament contained within the tubular main body; the data are readable by an electronic reader of the medicament delivery device 1 when the disposable cassette is in an attached position in the medicament delivery device 1.

Further, the disposable cassette 4 includes at least one distally extending protrusion 28 that extends distally away from a distal end 4b of the tubular main body 11. The at least one distally extending protrusion 28 comprises a distally facing surface 29. The distally extending protrusion 28 each comprises a distally facing inclined surface 36 inclined with respect to a longitudinal axis 102 of the tubular main body. The distally facing inclined surface 36 is connected with the distally facing surface 29 at an oblique angle.

In some possible embodiments, the activation ring 27 comprises two distally extending protrusion 28 symmetrically arranged with respect to the longitudinal axis 102 of the tubular main body 11.

A further protrusion 33 is arranged on the radially outwards facing surface of the tubular main body 11. The further protrusion 33 is located proximally with respect to the radially extending protrusion 19. Here the protrusion is an annular protrusion 33 that extend around the circumference, preferably the entire circumference of the tubular main body 11. The further protrusion 33 is configured to abut against the proximal end of the receiving interface 10 to limit the insertion length of the disposable cassette 4 in the receiving interface 10. In other words, the annular protrusion 33 acts like stop for preventing the cassette 4 to be inserted too deep into the receiving interface 10.

In this embodiment, a plunger rod adapter 35 is fitted in the tubular main body 11. The plunger rod adapter is cylindrical and is adapted to be pushed proximally by a plunger rod of the medicament delivery device during a medicament delivery event. The proximal motion of the plunger rod adapter 35 cause expulsion of a medicament from the medicament container 13.

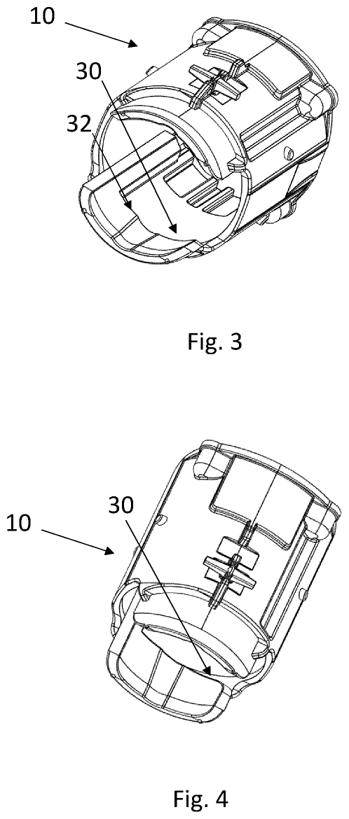

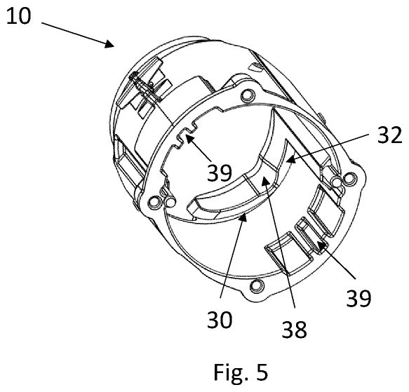

FIGS. 3 to 5 are different perspective views of a proximal receiving interface 10 of the medicament delivery device 1. The proximal receiving interface 10 is adapted to receive the cassette 4.

The proximal receiving interface 10 comprising a curved distally facing surface 30 adapted to receive and slidably guide the radially extending protrusion 19 of the cassette 4 in a distal direction when the cassette axially rotates relative to the receiving interface 10. In other words, the disposable cassette 4 is first axially inserted into the receiving interface 10. During a subsequent axial rotation of the disposable cassette 4, the radially extending protrusion 19 slides on the curved distally facing surface 30 of the receiving interface 10 of the medicament delivery device 1. The protrusion 19 slides on the curved surface 30 until the proximally facing surface 23 engages with a distally facing surface 32 of the receiving interface 10 to longitudinally lock the disposable cassette 4 in an attached position in the receiving interface 10.

The disposable cassette 4 shown in FIG. 2A comprises a second set of radially outwards extending protrusions 34 on the radially outwards facing surface 21 that are configured to engage in corresponding radially inner facing slots 38 of the receiving interface 10 to rotationally lock the disposable cassette 8 in the receiving interface 10. Preferably, the second set of radially extending protrusions 34 are longitudinal ribs.

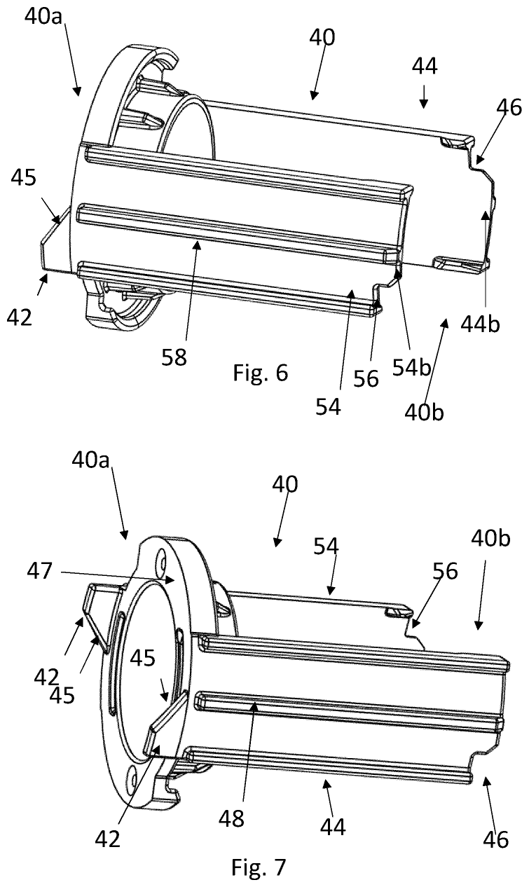

FIGS. 6 and 7 are two perspective view of an activation interface 40 according to embodiments of the present disclosure. The activation interface 40 comprises a proximal end 40a and a distal end 40b. The proximal end 40a comprises protrusions 42 that extend in a proximal direction. The protrusions 42 are configured to receive distally extending protrusion 29 of the cassette 4 when the cassette 4 is rotated as will be discussed further herein. The protrusions 42 comprise an inclined surface 45 adapted to receive the distally extending protrusion 36 of the disposable cassette 4 when the disposable cassette is rotated axially from its attached position.

The activation interface 40 comprises a first 44 and second distally extending arms 54. The first 44 and second distally extending arms 54 each comprise a cut-out 46, 56 on their respective distal ends 44b, 54b arranged diagonally with respect to each other so that the arms 44, 54 are asymmetric.

The activation interface further comprises longitudinal ribs 48 and 58 on the respective first and second arm 44, 54, that are configured to fit in and slide in corresponding slots or grooves 39 on a radially inwards facing surface of the distal end of the receiving interface 10.

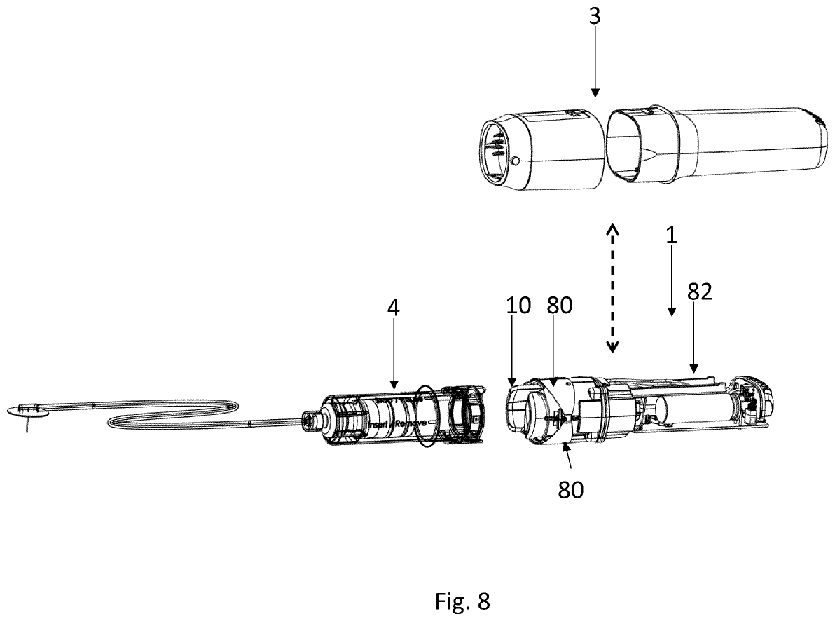

FIG. 8 is a partly exploded view of the medicament delivery device 1 with the housing 3 removed. A set of antennas 80 are shown that are configured to capture signals from the electronic tag 25. Further, a power pack 82 with electronic circuitry and batteries are arranged at the rear end of the medicament delivery device 1.

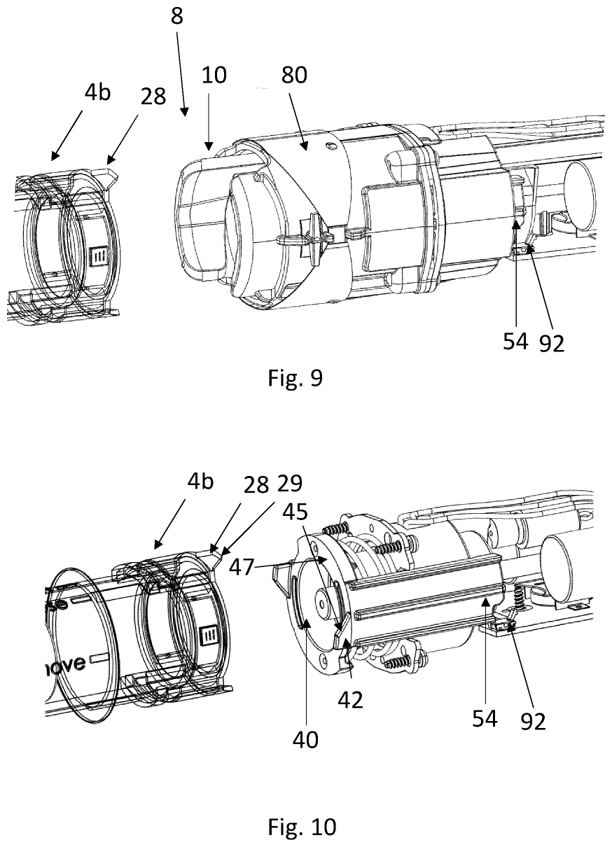

FIG. 9 is a close-up view of the distal end 4b of the disposable cassette 4 and the receiving interface 10 before the disposable cassette 4 is inserted in the receiving interface 10. Further, the second arm 54 of the activation interface 40 extends towards a second switch 92. On the opposite side, as is better seen in subsequent drawings, the first arm 44 extend towards a first switch 90. To better see the activation interface 40, the receiving interface 10 is removed in FIG. 10. Note that this is still prior to inserting the disposable cassette 4 in the receiving interface 10. A subassembly 8 of herein disclosed embodiments comprise the activation interface 40 and the receiving interface 10.

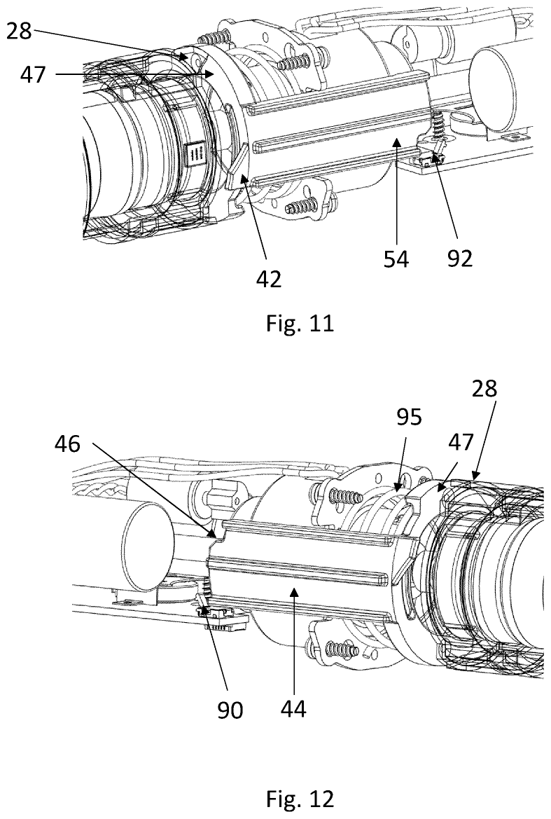

In FIGS. 11 and 12, the disposable cassette 4 is inserted in the receiving interface 10 but not yet mounted by rotating the disposable cassette 4 in the receiving surface 10 as discussed in relation to FIGS. 3-5. The arms 44 and 54 extend towards a respective first 90 and second switch 92. Further, a spring 95 is arranged to spring load the activation interface 40 away from the first switch 90 and the second switch 92.

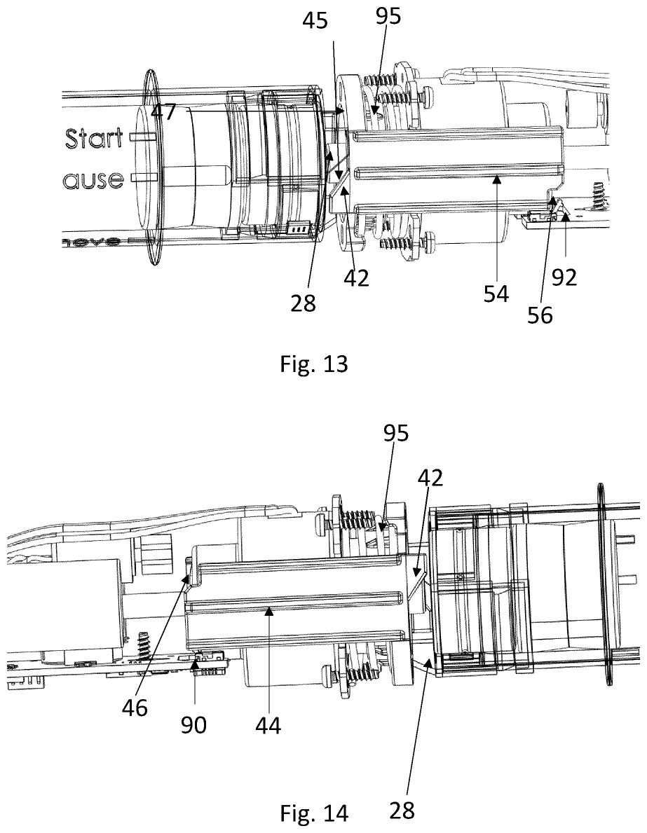

Turning to FIGS. 13 and 14, here with the disposable cassette 4 inserted in the receiving interface 10 and mounted by rotating the disposable cassette 4 in the receiving surface 10 as discussed in relation to FIGS. 3-5. As discussed, during the axial rotation of the disposable cassette 4, the radially extending protrusion 19 slides on the curved distally facing surface 30 of the receiving interface 10. This sliding motion on the curved surface 30 causes a distal axial motion of the disposable cassette 4 so that the distally facing surface 29 of the protrusions 28 abut against and push on a proximally facing surface 47 the activation interface 40 in a distal direction to the position shown in FIGS. 13 and 14. The axial rotation for locking of disposable cassette 4 may be about 60 degrees with respect to the receiving interface 10.

In FIG. 13 one side of the device 1 is shown where the second arm 54 of the activation interface 40 is shown to not reach the second switch 92 which is not activated by only mounting the disposable cassette in the receiving interface 10. However, the first arm (better seen in FIG. 14), having its cut-out 46 asymmetrically arranged with respect to the cut-out 56 of the second arm 54, reaches the first switch 90 to thereby active the first switch 90. Activation of the first switch 90 causes activation of the electronic reader of the circuitry 82 to read the electronic tag 25. The spring 95 is compressed by the axial motion of the activation interface 40 causes by the force from the disposable cassette 4 moving distally when mounted in the receiving interface 10.

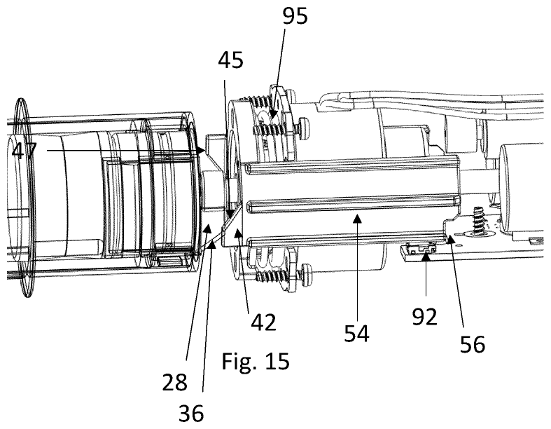

Turning to FIG. 15 where the disposable cassette has been further rotated in the receiving interface 10. The distally facing inclined surface 36 is arranged to interact with the surface 45 of the activation interface 40 when the disposable cassette 4 is rotated further. The interaction causes the activation interface 40 to move further distally. The cut-out 56 at the distal end 54b of the second arm 54 reach and activate the second switch 92 when the activation interface 40 moved distally. The activation of the second switch actuates a plunger rod of the medicament delivery device for initiating a priming action of the medicament container.

A rotation of the disposable cassette 4 in the opposite direction causes the activation interface 40 to move proximally by the force applied by the spring 95. This can cause deactivation of the first 90 and second switch 92 depending on degree of rotation of the disposable cassette 4 that allow the activation interface 40 to move variable distances in the proximal direction. A further rotation in this opposite direction allows for dismounting the cassette 4 from the receiving interface 10 so that it can be disposed of. Subsequently, a new cassette 4 can be mounted to the receiving interface 10 as described herein.

A medicament delivery device (such as an autoinjector) may generally include various other components. For example, a sensor unit which may recognize medicament delivery events, such as the needle inserted into an attachment portion of e.g., a pad, injection started, and medicament delivery operation ends, a memory unit which is configured to store the recorded data during the medicament delivery operation, a connectivity unit configured to transmit the stored data to a smart device or the network directly, a processing unit configured to control the entire system and processes the data before transmitting it, and/or user interface units that are configured to provide feedback to the patient, such as status LEDs, haptic, and/or audio feedback.

When the medicament delivery device is placed into the attachment portion, the sensors inside of the pad are configured to recognize the event and give feedback to the patient via haptic/visual or audio elements.

When the medicament delivery finishes, the sensors are configured to recognize the event and give feedback to the patient again. Further, the collected data is stored in the memory unit and may be transmitted to the smart device/network via the connectivity unit after the medicament delivery event finishes.

The sensor can be one of or the combination of the following: a mechanical switch, a Hall-effect sensor, an accelerometer.

The mechanical switch, hall-effect sensor, or accelerometer can be used for detection of the insertion of the a needle into an injection port.

The accelerometer can be used for detecting medicament delivery events.

Possible wireless communication methods include Bluetooth and Cellular Networks.

Bluetooth connectivity requires a smart device to transmit the stored data to the network and it requires a pairing action between the pad and the smart device before being able to use the supporting pad in case of 2-way connection. But it's a cheaper alternative and it requires less space on PCB. A 1-way connection does not require pairing.

The cellular network does not require any pairing process, it can be used as a plug-n-play device, no prior setup is needed, but it's more expensive and it requires more space on PCB.

Depending on the requirements of the product any of those two technologies can be used.

Such processing units may comprise a logic circuit or control unit including a microprocessor, microcontroller, programmable digital signal processor or another programmable device. The processing circuitry may also, or instead, each include an application specific integrated circuit, a programmable gate array or programmable array logic, a programmable logic device, or a digital signal processor. Where the processing circuitry includes a programmable device such as the microprocessor, microcontroller or programmable digital signal processor mentioned above, the processor may further include computer executable code that controls operation of the programmable device.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies.

Exemplary types of drugs that could be included in the medicament delivery devices described herein include, but are not limited to, antibodies, proteins, fusion proteins, peptibodies, polypeptides, pegylated proteins, protein fragments, protein analogues, protein variants, protein precursors, and/or protein derivatives. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g.

Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

The inventive concept has mainly been described above with reference to a few examples. However, as is readily appreciated by a person skilled in the art, other embodiments than the ones disclosed above are equally possible within the scope of the inventive concept, as defined by the appended claims.

Some aspects of the invention are defined by the following clauses.

1. A disposable cassette (4) configured to accommodate a medicament container (13), the cassette is attachable to a medicament delivery device (1) for delivery of the medicament to a medicament delivery site via an infusion set (6) attachable to the cassette, the disposable cassette (4) comprising:
a tubular main body (11) adapted to receive the medicament container (13) radially inside the tubular main body (11), the tubular main body having a through hole (15) at a proximal end (4a) through which an adapter (17) of the infusion set is attachable to the medicament container,
wherein the tubular main body comprises a radially extending protrusion (19) on a radially outwards facing surface, the radially extending protrusion includes a proximally facing surface (23),
wherein the disposable cassette is attachable to a proximal end (1a) of the medicament delivery device (1) by insertion of the disposable cassette (4) in a proximal receiving interface (10) of the medicament delivery device (1), and a subsequent axial rotation of the disposable cassette, whereby the radially extending protrusion (19) is configured to slide on a curved distally facing surface (30) of the receiving interface of the medicament delivery device until the proximally facing surface (23) engages with a distally facing surface (32) of the receiving interface (10) to longitudinally lock the disposable cassette (4) in an attached position in the receiving interface.

2. The disposable cassette according to clause 1, comprising an electronic tag (25) attached to a distal end (4b) of the tubular main body, the electronic tag having stored data that is related to the medicament contained within the main body; wherein the data are readable by an electronic reader (80) of the medicament delivery device when the disposable cassette is in the attached position.

3. The disposable cassette according to any one of clauses 1 and 2, comprising at least one distally extending protrusion (28) that extend distally away from the distal end (4b) of the tubular main body, wherein the at least one distally extending protrusion (28) comprise a distally facing surface (29) that is configured to abut against and push an activation interface (40) of the medicament delivery device (1) in a distal direction to cause actuation of a first switch (90) that activates the electronic reader.

4. The disposable cassette according to clause 3, wherein the distally extending protrusion (28) comprises a distally facing inclined surface (36) inclined with respect to a longitudinal axis (102) of the tubular main body, wherein the distally facing inclined surface (36) is arranged to interact with a surface (45) of the activation interface when the disposable cassette is rotated further.

5. The disposable cassette according to clause 4, wherein the interaction between the distally facing inclined surface (36) and the surface (45) of the activation interface cause the activation interface (40) to move distally.

6. The disposable cassette according to clause 5, wherein the activation interface activates a second switch (92) when it is moved distally by the interaction.

7. The disposable cassette according to any one of the preceding clauses, comprising a second set of radially extending protrusions (34) on the radially outwards facing surface (21) that are configured to engage in corresponding radially inner facing slots (38) of the receiving interface to rotationally lock the disposable cassette in the receiving interface.

8. The disposable cassette according to clause 7, wherein the second set of radially extending protrusions are longitudinal ribs (34).

9. The disposable cassette according to any one of the preceding clauses, comprising an activation ring (27) removably attached radially inside the tubular main body (1), the activation ring comprises the at least one distally extending protrusion (28).

10. The disposable cassette according to clauses 2 and 9, wherein the electronic tag (25) is attached to the activation ring (27).

11. The disposable cassette according to any one of clauses 9 and 10, wherein the activation ring (27) comprises two distally extending protrusion symmetrically arranged with respect to a longitudinal axis of the tubular main body.

12. The disposable cassette according to any one of the preceding clauses, comprising a removable tamper seal that seals the through hole (15) at the proximal end of the tubular main body.

13. The disposable cassette according to any one of the preceding clauses, comprising an annular cavity between the tubular main body and the medicament container at the proximal end of the tubular main body, wherein the annular cavity is configured to receive a flange (22) of the adapter (17) of the infusion set (6).

14. The disposable cassette according to clause 13, wherein the tubular main body comprises longitudinal ribs (18) facing radially inwards in the cavity, the longitudinal ribs being adapted to rotationally lock the adapter.

15. The disposable cassette according to any one of the preceding clauses, comprising a further protrusion (33) on the radially outwards facing surface of the tubular main body, the further protrusion is located proximally with respect to the radially extending protrusion (19), the further protrusion being configured to abut against the receiving interface (10) to limit the insertion length of the disposable cassette in the receiving interface.

16. The disposable cassette according to any one of the preceding clauses, wherein the attachment between the disposable cassette and the receiving interface is a bayonet coupling.

18. An infusion device (100) comprising a medicament delivery device and a disposable cassette according to any one of the preceding clauses.

19. A subassembly (2) for a medicament delivery device comprising:
a proximal receiving interface (10) adapted to receive a cassette accommodating a medicament container, the proximal receiving interface comprising a curved distally facing surface (30) adapted to receive and slidably guide a radially extending protrusion (19) of the cassette in a distal direction when the cassette axially rotates relative the receiving interface until a proximally facing surface (23) of the cassette engages with a distally facing surface (32) of the receiving interface to longitudinally lock the disposable cassette in an attached position in the receiving interface,
an activation interface (40) comprising a proximally facing surface (47) adapted to receive a distally extending protrusion (28) of the cassette, wherein when the activation interface (40) is pushed distally by the cassette it causes activation of a first switch (90) of the subassembly to activate an electronic reader to read an electronic tag of the cassette.

20. The subassembly according to clause 19, wherein the activation interface comprises a first (44) and second (54) distally extending arms extending towards a respective first (90) and second switch (92), where the first arm reaches and causes the activation of the first switch when the activation interface is moved distally.

21. The subassembly according to clause 20, wherein the activation interface comprises an inclined surface (45) adapted to receive the distally extending protrusion (28) of the cassette when the cassette is further rotated in the receiving interface (10), whereby an interaction between the distally facing surface of the cassette and the inclined surface of the activation interface cause the activation interface to move distally.

22. The subassembly according to clause 21, wherein the distal motion of the annular activation interface (40) causes the second arm (54) to reach and active the second switch (92).

23. The subassembly according to any one of clauses 20 to 22, wherein distal ends of the first arm and the second arm are asymmetric.

24. The subassembly according to any one of clauses 19 to 23, comprising a spring (95) arranged to spring load the activation interface away from the first switch and the second switch.

25. A medicament delivery device (1) comprising the subassembly according to any one of clauses 19 to 24.

1-15. (canceled)

16. A subassembly for a medicament delivery device, the subassembly comprising:
a proximal receiving interface adapted to receive a cassette accommodating a medicament container of the medicament delivery device, the proximal receiving interface comprising a curved distally facing surface adapted to receive and slidably guide a radially extending protrusion of the cassette in a distal direction when the cassette axially rotates relative the receiving interface until a proximally facing surface of the cassette engages with a distally facing surface of the receiving interface to longitudinally lock the cassette in an attached position in the receiving interface; and
an activation interface comprising a proximally facing surface adapted to receive a distally extending protrusion of the cassette, wherein when the activation interface is pushed distally by the cassette it causes activation of a first switch of the subassembly to activate an electronic reader to read an electronic tag of the cassette.

17. The subassembly according to claim 16, wherein the activation interface comprises a first and second distally extending arms extending towards a respective first and second switch, where the first arm reaches and causes the activation of the first switch when the activation interface is moved distally.

18. The subassembly according to claim 17, wherein the activation interface comprises an inclined surface adapted to receive the distally extending protrusion of the cassette when the cassette is further rotated in the receiving interface, whereby an interaction between the distally facing surface of the cassette and the inclined surface of the activation interface cause the activation interface to move distally.

19. The subassembly according to claim 18, wherein the distal motion of the annular activation interface causes the second arm to reach and active the second switch.

20. The subassembly according to claim 17, wherein distal ends of the first arm and the second arm are asymmetric.

21. The subassembly according to claim 16, further comprising:
a spring arranged to spring load the activation interface away from the first switch and the second switch.

22. The subassembly according to claim 16, wherein the cassette is configured to accommodate a medicament container, wherein the cassette is attachable to a medicament delivery device for delivery of the medicament to a medicament delivery site via an infusion set attachable to the cassette, the cassette further comprising:
a tubular main body adapted to receive the medicament container radially inside the tubular main body, the tubular main body having a through hole at a proximal end through which an adapter of the infusion set is attachable to the medicament container,
wherein the tubular main body comprises the radially extending protrusion on a radially outwards facing surface, the radially extending protrusion includes the proximally facing surface,
wherein the disposable cassette is attachable to a proximal end of the medicament delivery device by insertion of the disposable cassette in a proximal receiving interface of the medicament delivery device, and a subsequent axial rotation of the disposable cassette, whereby the radially extending protrusion is configured to slide on a curved distally facing surface of the receiving interface of the medicament delivery device until the proximally facing surface engages with the distally facing surface of the receiving interface to longitudinally lock the disposable cassette in an attached position in the receiving interface.

23. The subassembly according to claim 22, wherein the cassette comprises an electronic tag attached to a distal end of the tubular main body, the electronic tag having stored data that is related to the medicament contained within the main body, and wherein the data are readable by an electronic reader of the medicament delivery device when the disposable cassette is in the attached position.

24. The subassembly according to claim 22, wherein the cassette is disposable.

25. The subassembly according to claim 22, wherein the cassette comprises a second set of radially extending protrusions on the radially outwards facing surface that are configured to engage in corresponding radially inner facing slots of the receiving interface to rotationally lock the disposable cassette in the receiving interface.

26. The subassembly according to claim 22, wherein the second set of radially extending protrusions are longitudinal ribs.

27. The subassembly according to claim 22, wherein the cassette comprises an activation ring removably attached radially inside the tubular main body, and wherein the activation ring comprises the at least one distally extending protrusion.

28. The subassembly according to claim 22, wherein the electronic tag is attached to the activation ring.

29. The subassembly according to claim 22, wherein the cassette comprises an annular cavity between the tubular main body and the medicament container at the proximal end of the tubular main body, and wherein the annular cavity is configured to receive a flange of the adapter of the infusion set.

30. A medicament delivery device comprising the subassembly according to claim 16.
